SEQUENCE LISTING (6 sequences)

SEQ ID NO: 1 - DNA, 51 bp, synthetic construct
  misc_feature 1..51: note = 5'UTRgRNA_TGGT1_253780 primer
  source 1..51: mol_type = other DNA
tggggatgtcaagttgtgttttccgtgatcttaacagttttagagctagaa

SEQ ID NO: 2 - DNA, 51 bp, synthetic construct
  misc_feature 1..51: note = 3'UTRgRNA_TGGT1_253780 primer
  source 1..51: mol_type = other DNA
tggggatgtcaagttgttacctcttcttctgcacgggttttagagctagaa

SEQ ID NO: 3 - DNA, 60 bp, synthetic construct
  misc_feature 1..60: note = mNG_gch_overhangs_5UTR primer
  source 1..60: mol_type = other DNA
acttctgggtgaaccagcgggaggcgggcgtgcaaaagcaccatgcatgtcccgcgttcg

SEQ ID NO: 4 - DNA, 60 bp, synthetic construct
  misc_feature 1..60: note = mNG_gch_overhangs_3UTR primer
  source 1..60: mol_type = other DNA
gggaatgctgtgaagttcagtttgacattcggcgccacaggtgtcactgtagcctgccag

SEQ ID NO: 5 - DNA, 24 bp, synthetic construct
  misc_feature 1..24: note = TGGT1_253780_F primer
  source 1..24: mol_type = other DNA
ccccacataaaagtctgttgggct

SEQ ID NO: 6 - DNA, 19 bp, synthetic construct
  misc_feature 1..19: note = TGGT1_253780_R primer
  source 1..19: mol_type = other DNA
gcattgtgggctgtgaccc

(19) United States
(12) Patent Application Publication
(10) Pub. No.: US 2025/0295658 A1
(43) Pub. Date: Sep. 25, 2025

(54) ANTIPARASITIC AGENTS AND METHODS

(71) Applicant: Whitehead Institute for Biomedical Research, Cambridge, MA (US)

(72) Inventors: Christopher Joseph Giuliano, Cambridge, MA (US); Sebastian Lourido, Cambridge, MA (US)

(21) Appl. No.: 18/862,816

(22) PCT Filed: May 4, 2023
(86) PCT No.: PCT/US2023/066592

§ 371 Date: Nov. 4, 2024

Related U.S. Application Data
(60) Provisional application No. 63/339,281, filed on May 6, 2022.

(51) Int. Cl. A61K 31/505; A61K 31/522; A61K 31/635; A61P 33/02; G01N 33/50

(52) U.S. Cl. CPC A61K 31/505; A61K 31/522; A61K 31/635; A61P 33/02; G01N 33/5014; G01N 33/5085; G01N 2333/43526

(57) ABSTRACT

Methods of reducing viability of a parasite in a subject involve administering an effective amount of an inhibitor of guanosine-5′-triphosphate cyclohydrolase I (GCH) to the subject. Methods of identifying an agent that reduces viability of an apicomplexan parasite involve culturing an apicomplexan parasite in a cell culture; adding an agent to the cell culture; and detecting a concentration of one or more of 7,8-dihydroncopterin triphosphate, 6-pynivoyl-tetrahydropterin, tetrahydrobiopterin, tetrahydrofolate, folate, dihydrofolate, or dihydrobiopterin in the cell culture after a period of time.

### RELATED APPLICATION

This application claims the benefit of U.S. Provisional Application No. 63/339,281, filed on May 6, 2022. The entire teachings of the above application are incorporated herein by reference.

### GOVERNMENT SUPPORT

This invention was made with government support under R01 AI158501 from the National Institutes of Health. This invention was made with government support under R01 AI144369 from the National Institutes of Health. The government has certain rights in the invention.

### Incorporation by Reference of Material in XML

This application incorporates by reference the Sequence Listing contained in the following extensible Markup Language (XML) file being submitted concurrently herewith:
a) File name: 03992068001.xml; created Apr. 18, 2023, 12,288 Bytes in size.

### BACKGROUND

The phylum Apicomplexa includes many parasitic protists that are found in a wide variety of environments, including soil, freshwater, and marine habitats. Many of these parasites cause human and veterinary diseases, including malaria, babesiosis, toxoplasmosis, neosporosis, and cryptosporidiosis. Existing treatments for these diseases, such as pyrimethamine, can cause toxicity. Additionally, drug resistance is emerging for a number of frontline antiparasitic drugs. Accordingly, new methods of treating infections by apicomplexan parasites are needed.

### SUMMARY

Described herein are experiments demonstrating the unexpected discovery that inhibitors of guanosine-5′-triphosphate cyclohydrolase I (GCH) can reduce viability of an apicomplexan parasite.

Described herein are methods relating to reducing viability of an apicomplexan parasite in a subject in need thereof. The methods include administering an effective amount of an inhibitor of guanosine-5′-triphosphate cyclohydrolase I (GCH) to the subject.

Described herein are methods of identifying an agent that reduces viability of a parasite. In general, these methods involve: culturing an apicomplexan parasite in a cell culture; adding an agent to the cell culture; detecting a concentration of one or more of 7,8-dihydroneopterin triphosphate, 6-pyruvoyl-tetrahydropterin, tetrahydrobiopterin, tetrahydrofolate, folate, dihydrofolate, and dihydrobiopterin in the cell culture after a period of time. A decrease in concentration of one or more of 7,8-dihydroneopterin triphosphate, 6-pyruvoyl-tetrahydropterin, tetrahydrobiopterin, tetrahydrofolate, folate, dihydrofolate, and dihydrobiopterin indicates that the agent reduces viability of the apicomplexan parasite.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a graph showing results of “phenotype score” indicating the relative fitness of Toxoplasma mutants.

FIG. 1B is a graph comparing phenotype scores for parasites grown in HPLM against parasites grown in DMEM.

FIG. 1C is an illustration showing GCH at the top of a complex metabolic network.

FIG. 1D is a chart showing that Δgch parasites have a fitness defect in HPLM media, which can be rescued by the addition of exogenous folate or BH4 to HPLM media.

FIG. 1E is a chart showing that Δgch parasites are completely attenuated during mouse infection, causing no deaths in infected mice. The result provides genetic evidence that a chemical inhibitor of GCH would be of value as an antiparasitic therapeutic.

FIG. 1F is a set of plaque assays showing the antiparasitic activity of DAHP, and rescue of this activity with exogenous BH4.

FIG. 1G is a graph of percent lysis of host cells by parasites when grown in pyrimethamine with and without 2.5 mM DAHP.

FIG. 1H is a chart of the IC₅₀ for pyrimethamine compared to pyrimethamine supplemented with 2.5 mM DAHP.

FIG. 2A is a dose sensitivity curve of Plasmodium falciparum to pyrimethamine in the absence or presence of DAHP.

FIG. 2B is a chart showing IC₅₀ values of Plasmodium falciparum to pyrimethamine with and without DAHP co-treatment.

FIG. 2C is a dose sensitivity curve of Plasmodium falciparum to atovaquone in the absence or presence of DAHP.

FIG. 2D is a chart showing IC₅₀ values of Plasmodium falciparum to atovaquone with and without DAHP co-treatment.

FIG. 2E is a chart showing population doublings of C57B1/6J mouse embryonic fibroblasts in the presence and absence of DAHP.

FIG. 2F is a diagram of the engineered parasite strain allowing for bradyzoite induction including a firefly luciferase reporter for CST10 (bradyzoite marker) and a nano luciferase reporter for SAG1 (tachyzoite marker).

FIGS. 2G-H are charts showing CST10 (bradyzoite) (FIG. 2G) and SAG1 (tachyzoite) (FIG. 2H) luciferase reporter signals during bradyzoite induction by shield-1.

FIGS. 2I-K are charts showing dynamics of parasite state (tachyzoite vs. bradyzoite) after shield-1 washout. Conditions include parasite samples never induced with shield-1 (−), samples maintained in shield-1 for the duration of the experiment (+), and samples induced with shield-1 for 48 hr before washout (washout). FIG. 2I: 24 hours post initial shield-1. FIG. 2J: 48 hours post initial shield-1. FIG. 2K: 72 hours post initial shield-1.

### DETAILED DESCRIPTION

A description of example embodiments follows.

### Methods of Reducing Viability of Apicomplexan Parasites

Described herein are methods relating to reducing viability of an apicomplexan parasite in a subject in need thereof. The methods include administering an effective amount of an inhibitor of guanosine-5′-triphosphate cyclohydrolase I (GCH) to the subject.

Inhibitors of GCH are disclosed in U.S. Pat. No. 7,906,520, which is incorporated by reference in its entirety. Examples of inhibitors of GCH are: 2,4-diamino-6-hydroxypyrimidine (DAHP); 4-chloro-2,6-diaminopyrimidine; 4-bromo-2,6-diaminopyrimidine; 4-iodo-2,6-diaminopyrimidine; 5-chloro-2,4-diamino-6-hydroxypyrimidine; 5-bromo-2,4-diamino-6-hydroxypyrimidine; 5-iodo-2,4-diamino-6-hydroxypyrimidine; 2,4,5-triamino-6-hydroxypyrimidine; guanine; 8-bromoguanine; 8-hydroxyguanine; 8-methylguanine; 8-mercaptoguanine; and 8-azaguanine.

Many apicomplexan parasites are known, include those of the Toxoplasma genus (e.g., Toxoplasma gondii), the Plasmodium genus (e.g., Plasmodium vivax, Plasmodium falciparum, Plasmodium malariae, Plasmodium ovale, or Plasmodium knowlesi), the Hammondia genus, the Neospora genus (e.g., Neospora caninum), the Sarcocystis genus (e.g., Sarcocystis neurona), the Theileria genus, the Eimeria genus, the Babesia genus, and the Cryptosporidium genus.

In some embodiments, the methods include administering an inhibitor of dihydrofolate reductase (DHFR), such as pyrimethamine, to the subject. In some embodiments, the method reduces the IC₅₀ of the inhibitor of DHFR (e.g., pyrimethamine) by at least 10%, at least 20%, at least 30%, at least 40%, at least 50%, at least 60%, at least 70%, at least 80%, or at least 90%. Reducing the IC₅₀ of pyrimethamine may allow for administration of smaller doses and/or a decrease in the dosing rate, which may reduce the incidence of known side effects associated with pyrimethamine. In some embodiments, the apicomplexan parasite is resistant to the inhibitor of DHFR (e.g., pyrimethamine).

In some embodiments, the methods include administering an inhibitor of dihydropteroate synthase (DHPS), such as sulfadiazine, to the subject. In some embodiments, the method reduces the IC₅₀ of the inhibitor of DHPS (e.g., sulfadiazine) by at least 10%, at least 20%, at least 30%, at least 40%, at least 50%, at least 60%, at least 70%, at least 80%, or at least 90%. Reducing the IC₅₀ of sulfadiazine may allow for administration of smaller doses and/or a decrease in the dosing rate, which may reduce the incidence of known side effects associated with sulfadiazine. In some embodiments, the apicomplexan parasite is resistant to inhibitor of DHPS (e.g., sulfadiazine).

The methods described herein are applicable to a variety of subjects, including humans and animals. Some animals are more susceptible to infection by particular types of parasites. In some embodiments, the animal is a cat (which can be susceptible to infection by Toxoplasma). In some embodiments, the animal is cattle (which can be susceptible to infection by Toxoplasma or Neospora). In some embodiments, the animal is a pig (which can be susceptible to infection by Toxoplasma). In some embodiments, the animal is a chicken (which can be susceptible to infection by Toxoplasma or Eimeria). In some embodiments, the animal is a sheep (which can be susceptible to infection by Toxoplasma). In some embodiments, the animal is a goat (which can be susceptible to infection by Toxoplasma).

In some embodiments, the subject has toxoplasmosis. In some embodiments, the subject has babesiosis. In some embodiments, the subject has cryptosporidiosis. In some embodiments, the subject has malaria. In some embodiments, the methods are prophylactic and involve administering the compounds to subjects at risk of toxoplasmosis, such as subjects who are immunocompromised (e.g., patients with human immunodeficiency virus (HIV)). In some embodiments, the methods are prophylactic and involve administering the compounds to subjects at risk of malaria, such as subjects who are immunocompromised (e.g., patients with human immunodeficiency virus (HIV)).

In some embodiments, the inhibitor of GCH is administered at a rate from 50 mg/kg/day to 300 mg/kg/day.

The methods described herein are exemplified with respect to DAHP, but other inhibitors of GCH can be used.

### Methods of Identifying Agents That Reduce Viability of an Apicomplexan Parasite

Described herein are methods of identifying an agent that reduces viability of a parasite. In general, these methods involve: culturing a parasite in a cell culture; adding an agent to the cell culture; detecting a concentration of one or more of 7,8-dihydroneopterin triphosphate, 6-pyruvoyl-tetrahydropterin, tetrahydrobiopterin, tetrahydrofolate, folate, dihydrofolate, and dihydrobiopterin in the cell culture after a period of time. A decrease in concentration of one or more of 7,8-dihydroneopterin triphosphate, 6-pyruvoyl-tetrahydropterin, tetrahydrobiopterin, tetrahydrofolate, folate, dihydrofolate, and dihydrobiopterin indicates that the agent reduces viability of the apicomplexan parasite.

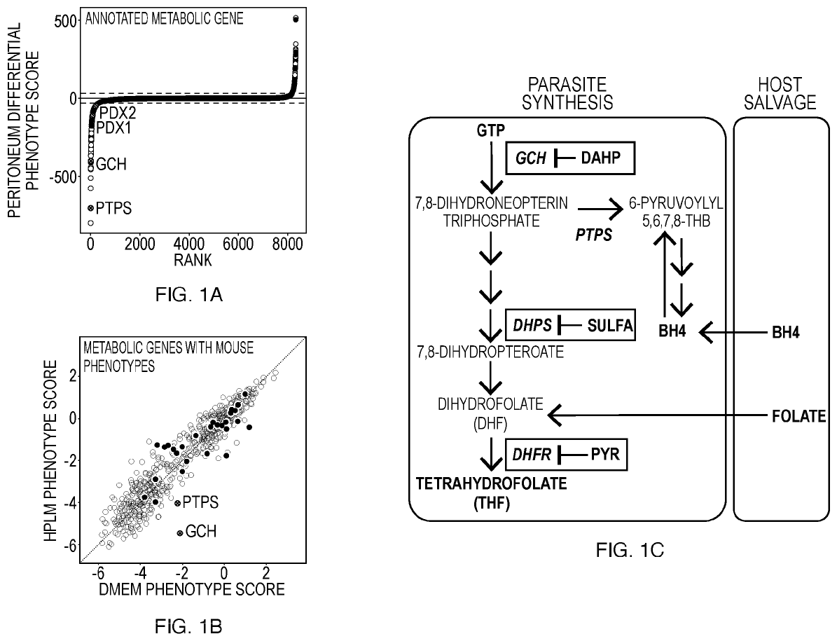

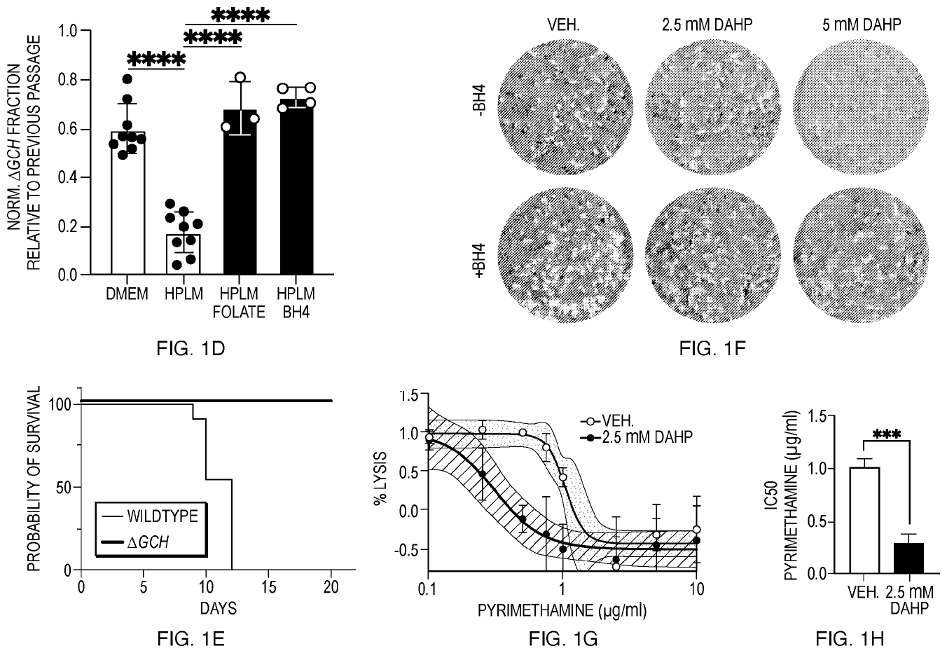

In some embodiments, the cell culture is a folate-poor cell culture condition (e.g., folate concentration less than about 1 mg/L). In some embodiments, the cell culture is a folate-rich cell culture condition (e.g., folate concentration greater than about 4 mg/L). In some embodiments, the cell culture is a BH4-poor cell culture condition (e.g., BH4 concentration less than about 1 μM). In some embodiments, the cell culture is a BH4-rich cell culture condition (e.g., BH concentration greater than about 50 μM). Methods in which the cell culture is deficient or poor in a nutrient can be used to determine on-target of activity of the agent in the GCH pathway (e.g., as described with respect to FIG. 1D and 1F).

In some embodiments, the apicomplexan parasite does not express GCH. In some embodiments, the methods include adding an antiparasitic compound to the cell culture.

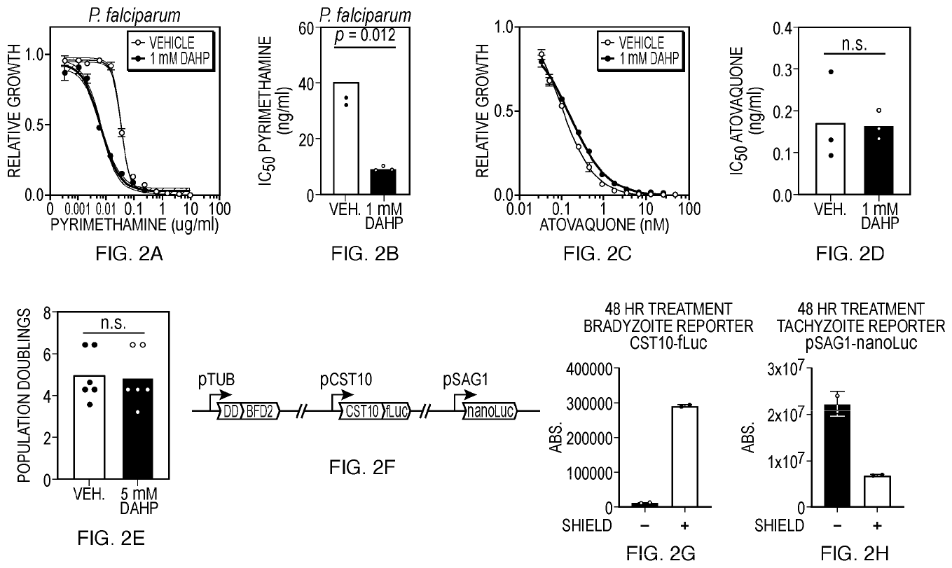

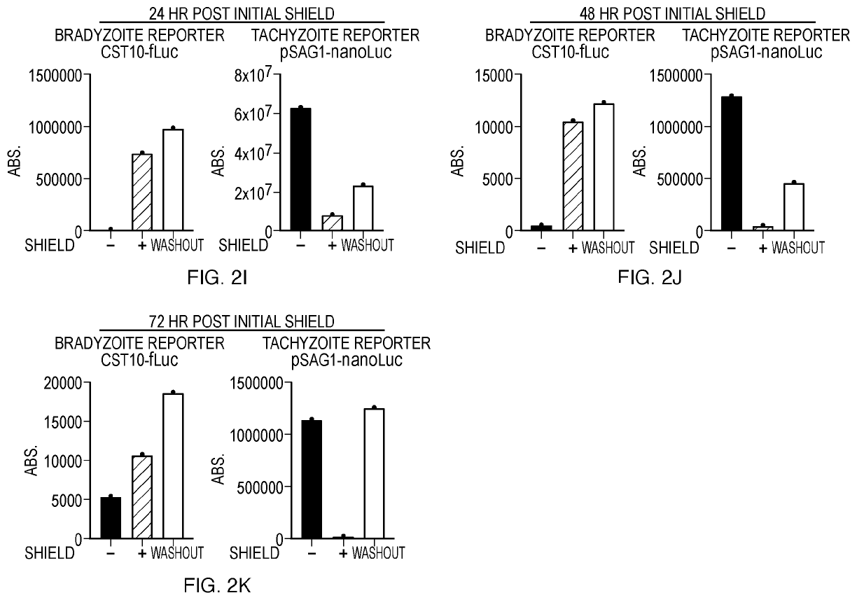

In some embodiments, the methods include the use of an engineered Toxoplasma strain capable of recapitulating and reporting on both the acute and chronic stages of the parasite by dual-luciferase assays (FIGS. 2F-K).

Reducing the IC₅₀ of the antiparasitic agent can indicate synergism of the agent and the antiparasitic agent. For example, the agent can reduce the IC₅₀ of the antiparasitic agent by at least 10%, at least 20%, at least 30%, at least 40%, at least 50%, at least 60%, at least 70%, at least 80%, or at least 90%.

### EXEMPLIFICATION

### Overview

CRISPR screening has been adapted to conduct a genome-wide screen of Toxoplasma during mouse infection. Screening parasites in mice can reveal features of parasite biology that are not easily captured in cell culture. Of particular interest are metabolic genes, as the nutrient environment of the mouse is distinct from that found in typical culture systems. The mouse nutrient environment would therefore place unique constraints on metabolic networks important for parasite growth. Throughout the Exemplification, the term parasite refers to experiments conducted on Toxoplasma gondii, except where otherwise noted.

### GTP Cyclohydrolase I is a Metabolic Dependency and a Druggable Anti-Parasitic Target

As an intracellular parasite, Toxoplasma is highly influenced by the nutrient environment of the host cytosol¹⁻³. In conducting a genome-wide CRISPR screen of Toxoplasma during mouse infection, the fitness of annotated metabolic genes between mouse and cell culture environments was surveyed. As shown in FIG. 1A, the screens generate a “phenotype score” for each gene of the Toxoplasma genome. Negative phenotype scores indicate that a gene is fitness-conferring. Phenotype scores can be compared from parasites in cell culture and mouse infection to identify genes that are differentially required for mouse infection. For example, PDX1 and PDX2 (TGGT1_237140, TGGT1_281490) were identified as fitness-conferring genes during mouse infection (FIG. 1A). PDX1 and PDX2 are required for the production of vitamin B6 precursors. Consistent with these screens, PDX1 knockouts were recently shown to be avirulent in mouse infection, despite remaining viable in cell culture¹.

The dependency of genes like PDX1 and PDX2 in mouse infection highlights how high nutrient concentrations in standard culture media (such as DMEM) can mask genetic vulnerabilities in the parasite. Previous work has attempted to generate models that reconcile the differences between culture and animal environments by formulating Human Plasma-Like Medium (HPLM), a media alternative that closely mirrors physiological nutrient concentrations⁴. To further understand the metabolic bottlenecks that arise in animal infection, HPLM was tested to determine whether it could recapitulate metabolic dependencies identified during mouse infection. To gain an unbiased view of parasite metabolism in this setting, metabolism-targeted CRISPR screens (focused on metabolic genes from the Toxoplasma genome) were conducted in standard (DMEM) and physiological-like (HPLM) culture conditions¹, and the results are shown in FIG. 1B. The majority of metabolic genes exhibited similar fitness scores in DMEM and HPLM, demonstrating that the metabolic bottlenecks experienced by parasites during animal infection cannot be easily captured in cell culture.

Unlike most metabolic genes however, two candidates, TGGT1_253780 and TGGT1 305800, were identified as highly fitness-conferring in both HPLM media and mouse infection. TGGT1_253780 encodes an ortholog of GTP Cyclohydrolase I (GCH), a highly conserved protein responsible for the conversion of GTP to 7,8-dihydroneopterin triphosphate. Additional enzymes facilitate the conversion of 7,8-dihydroneopterin triphosphate into tetrahydrobiopterin (BH4) and tetrahydrofolate (THF), two essential cofactors required for nucleic acid and amino acid metabolism^{5.6}. Highlighting the importance of this pathway, the second candidate, TGGT1_305800, encodes an ortholog of 6-pyruvoyl-tetrahydropterin synthase (PTPS), an enzyme immediately downstream of GCH⁷. While the ability to synthesize BH4 from GTP is shared between parasites and vertebrate hosts, parasites are distinct in their ability to utilize these pathways for THE synthesis. By contrast, vertebrates can only generate THE after obtaining folate, a THF-precursor, from their diet. In addition to internal synthesis, parasites are able to tap into the BH4 and THF supplies of their hosts through the uptake of BH2/BH4 and folate from the host cytosol (FIG. 1C)⁸⁻¹⁰.

Folate is present at lower concentrations in HPLM compared to DMEM^{4,11,12}. Under these folate-limiting conditions, the screen suggested that parasites cultured in HPLM may rely more heavily on the THF and BH4 synthesis pathway through GCH. To validate the screen results, a GCH-knockout (Δgch) parasite line was generated. The screen is confirmed by showing that Δgch parasites have a fitness defect in HPLM media. This defect could be rescued with the addition of exogenous folate and BH4 to the media, indicating that Δgch parasites become auxotrophic for folate and BH4 (FIG. 1D). The ability to rescue the fitness defect allows verification of the on-target activity of drugs inhibiting this pathway by growing parasites in HPLM (folate and BH4-poor) or HPLM with folate and BH4 supplementation (folate and BH4-rich). Interestingly, parasite synthesis of folate and BH4 is often described as two distinct pathways stemming from GCH. However, the ability of either exogenous folate or BH4 alone to rescue Δgch parasites indicates that there is sufficient reverse activity of these pathways to allow interconversion of these metabolites. As vertebrate hosts are unable to synthesize folate themselves, there is no potential for BH4 to be converted to folate in the host cytosol, indicating that the interconversion of BH4 and folate must take place within the parasite.

As diagramed in FIG. 1C, DHFR functions in the final stages of folate metabolism, acting downstream of the convergence of the folate synthesis and salvage pathways¹⁷. DHPS is traditionally thought to function solely in folate synthesis, but as shown herein, BH4 and folate are interconvertible in the parasite¹⁷. This then places DHPS downstream of BH4 uptake in the conversion of BH4 to folate. Therefore, pyrimethamine and sulfadiazine accomplish their antiparasitic effect by interrupting both synthesis and salvage supplies of folate for parasites. The results indicate, however, that under physiological conditions folate salvage alone is insufficient to support parasite growth. Indeed, Δgch parasites failed to cause infection in mice, despite having a fully intact folate salvage pathway (FIG. 1E). The result provides genetic evidence that a chemical inhibitor of GCH would be of value as an antiparasitic therapeutic. Therefore, despite acting solely in folate synthesis, GCH is elevated as a potential drug target in this classically drugged pathway.

The reliance of apicomplexan parasites on the folate pathway has been long appreciated. Current standard-of-care treatments for apicomplexan infections rely on inhibition of folate metabolism through administration of pyrimethamine and sulfadiazine, inhibitors of dihydrofolate reductase (DHFR) and dihydropteroate synthase (DHPS), respectively¹³. While these drugs remain the standard-of-care, long-term treatment often results in patient toxicity¹⁴. In the case of malaria, drug-resistance is emerging among Plasmodium parasites, underscoring the need for alternative treatment strategies^{13,15,16}. Given the role of GCH upstream of folate metabolism, chemical inhibition of GCH presents an alternative modality to antagonize this classically targeted pathway.

An inhibitor of GCH, 2,4-Diamino-6-hydroxyprimidine (DAHP), has been characterized in the literature for decades. Perturbation of GCH via DAHP has been utilized in a variety of studies including pain management, cancer, and cardiovascular disease¹⁸⁻²³. A single report from 1964 documents the use of DAHP against the unrelated protozoan Crithidia fasciculata²⁴, but DAHP has not been extensively studied as an anti-microbial nor proposed as a therapeutic for apicomplexan infections.

Despite poor solubility and a high turn-over rate²¹, FIG. IF shows that 5 mM DAHP is able to block the parasite lytic cycle, while 2.5 mM DAHP has no apparent effect on parasite viability. Despite the high concentrations of DAHP used in these assays, the antiparasitic effect of DAHP was rescued by exogenous BH4, supporting GCH as the functional target of this compound. DAHP has been extensively characterized for its inhibitory effect on human GCH^{25,26}. From these experiments, it is unclear whether the antiparasitic effect of DAHP is a result of direct inhibition of parasite GCH, inhibition of host GCH leading to reduced BH4 stores available for salvage, or a combination of both.

Recent clinical isolates of pyrimethamine-resistant Plasmodium parasites were shown to have copy number amplifications of GCH, suggesting that increased flux through the folate synthesis pathway may facilitate pyrimethamine resistance^{6,27}. Given the role of GCH upstream of folate metabolism, DAHP was tested to determine whether it produces a synergistic effect with standard-of-care antiparasitic treatments that target folate metabolism, such as pyrimethamine. DAHP proved to be highly synergistic when used in combination with pyrimethamine, resulting in a 70% reduction in the pyrimethamine IC₅₀ value (FIGS. 1G-H). The synergy of DAHP and pyrimethamine present an opportunity to circumvent cases of pyrimethamine resistance and reduce patient toxicity from standard pyrimethamine dosage regimens.

The antiparasitic synergy of DAHP with pyrimethamine is conserved in Plasmodium falciparum, as co-treatment resulted in a similar reduction in the pyrimethamine IC₅₀ value (FIGS. 2A-B). By contrast, DAHP had no potentiating effect when used in combination with atovaquone—an inhibitor of the mitochondrial electron transport chain with no direct relation to folate or BH4 metabolism³³ (FIGS. 2C-D). A 5 mM DAHP dose had no effect on the growth of a proliferative mouse fibroblast cell line, indicating that a therapeutic window may exist allowing for parasite inhibition while maintaining host viability (FIG. 2E).

To facilitate the identification and generation of more potent GCH inhibitors and antiparasitic compounds, a Toxoplasma strain optimized for chemical library screening was engineered (FIG. 2F). This strain is able to recapitulate the biology of both the acute tachyzoite stage as well as the chronic bradyzoite stage. Differentiation from the tachyzoite to bradyzoite stage is mediated through the expression of Bradyzoite Formation Deficient 2 (BFD2) tagged with a degradation domain. Under standard conditions, the degradation domain ensures BFD2 is degraded from parasites, maintaining them in the tachyzoite stage. Upon the addition of the small-molecule shield-1, BFD2 is stabilized and causes differentiation of parasites to the bradyzoite stage.³⁴ The engineered parasite strain allows the use of a dual-luciferase system to report on differentiation status and general parasite viability. Expression of firefly luciferase is driven by fusion via t2a peptide to the bradyzoite marker CST10, whereas expression of nano-luciferase is driven by the tachyzoite promoter SAG1. Addition of shield-1 is able to simultaneously increase the CST10 bradyzoite marker while decreasing the SAGI tachyzoite marker (FIGS. 2G-H). This engineered parasite strain exhibits sustained growth in the bradyzoite stage even after removal of shield-1 (FIGS. 2I-K). This sustained differentiation after washout of shield-1 indicates that the engineered bradyzoites are perpetuated by endogenous pathways after the initial differentiation induced by shield-1. This enables examination of the chronic parasite state with minimal interference on the endogenous circuity. Chemical screens of this engineered strain can therefore identify compounds that bias parasite state either to the tachyzoite or bradyzoite stage, as well as compounds capable of killing both stages simultaneously. The chronic bradyzoite stage is refractory to many antiparasitic drugs, and thus the ability to recapitulate this stage in culture is highly valuable in the pursuit of next generation antiparasitic compounds.

### Materials and Methods

### Genome-wide Screening in Mice

Briefly, gRNAs targeting the entirety of the parasite genome were cloned into an expression vector. gRNAs targeting all genes were divided into 17 sublibraries targeting approximately 500 genes each. In each mouse screen, 2 libraries were combined, to screen approximately 1000 genes in a single experiment. Briefly, screens were conducting by transfecting in gRNA libraries into Cas9-expressing parasites. Parasite populations were maintained in culture (DMEM) for four passages prior to splitting the population by injecting intraperitoneally (I.P.), or further maintenance in culture. 6 days post mouse infection, parasites were harvested from infected mice in their peritoneum, liver, spleen, lung, heart, and brain. Harvested parasites were expanded in cell culture until plate lysis to enrich for parasite genomic DNA. Genomic DNA was harvested from parasites through the Qiagen blood and tissue kit. gRNAs were amplified from genomic DNA, and sequenced on a Next Seq. gRNA abundance was calculated from sequencing, and used to determine fitness cost of each gene in cell culture and in mouse infection.

Genome-Wide Screening in DMEM vs. HPLM Media

Briefly, a gRNA library targeting annotated metabolic genes of Toxoplasma was cloned into a gRNA expressing vector. The gRNA library was transfected into Cas9-expressing parasites grown in DMEM. At first lysis (48 h), the parasite population was split to grow in either HPLM or DMEM. Parasites were cultured in either media condition for 3 passages. Genomic DNA was harvested from the parasites at each passage. gRNAs were amplified from parasite genomic DNA and sequenced on a MiSeq. gRNA abundances were used to calculate fitness scores of mutant parasites in DMEM and HPLM.

### Generation of Δgch Parasites

gRNAs targeting the 5′ and 3′ ends of TGGT1_253780 (GCH) were cloned into a Cas9-expressing vector containing a sgRNA scaffold. A mNeonGreen expression construct was PCR amplified with overhangs homologous to the regions immediately surrounding the cut sites of these gRNAs. The two gRNAs and the mNeonGreen repair template were transfected into RHAku80: nanoLuc parasites. At first lysis (48 h), transfectants were FACS sorted for mNeonGreen positive parasites. Parasites were then subcloned into 96 well plates. After 6 days of growth, wells containing single plaques were selected. Single plaques were expanded and PCR validated for GCH knockout.

### Assessing Δgch Parasite Virulence in Mice

Intracellular RHAku80 and Δgch strains maintained in DMEM+10% FBS were washed with PBS to remove serum. Intracellular parasites were then scrapped in PBS, syringe released, and filtered through a 5 μM filter. Parasites were then counted and diluted to 1e5 parasites/ml. 1e4 parasites in 100ul PBS were then injected I.P. into CD-1 female mice of 8-10 weeks of age. Mouse health was monitored daily after injection until the endpoint of the experiment (day 60 post injection). At least 4 mice were included in each group. Anti-Toxoplasma serum ELISAs were conducted to verify infection in surviving mice. Mouse survival plots were generated in Graphpad Prism 9.

### Growth Competitions in Various Media

Δgch (mNeonGreen-positive) and WT (dTomato-positive) parasites were syringe released and filtered through 5 μm filters. Parasites were then counted and 8e5 parasites of each strain were mixed together in DMEM+10% FBS. The mixed population was analyzed by MACSQuant to determine the precise starting ratio of parasites. 48 h later, parasites were force lysed, and 100-200 μl were passed to new 6 wells containing either DMEM or HPLM with 10% FBS. For BH4 rescue experiments, wells received either 50 um BH4 or the equivalent volume of DMSO vehicle. For folate rescue experiments, folate concentrations were raised to 30 mg/l in HPLM. MACSQuant readings were taken at a lysis time point (48 h post infection) to track population dynamics of Δgch parasites by relative abundance to WT dTomato+parasites.

Plaque Assays with DAHP and BH4

RHAku80 were maintained for at least two passages in DMEM and HPLM prior to beginning the plaque assay. Confluent human fibroblasts were maintained in 6-well plates in either DMEM or HPLM for at least 24 h prior to the start of the assay. To begin the experiment, parasites maintained in DMEM and HPLM were syringe released and filtered through a 5 μM filter. Parasites were then counted and diluted such that each well received 500 parasites. Wells were then treated with the indicated concentrations of DAHP or PBS vehicle. For the BH4 rescue experiments, wells received 50 μM BH4 or the equivalent volume of DMSO vehicle. Plaques were allowed to grow for 6 days prior to fixation. For fixation, wells were first washed with PBS, then fixed for 10 min at room temperature with 100% ethanol. Wells were then stained for 10 min with crystal violet (12.5 g crystal violet, 125 ml 100% ethanol, 500 ml 1% ammonium oxalate). Wells were then washed three times with water and allowed to dry overnight.

### Pyrimethamine/DAHP Drug Synergy Lytic Assays

96 well plates were utilized to generate 8-point dosage curves of parasites grown in various concentrations of pyrimethamine with and without 2.5 mM DAHP. At the start of the assay, prior to parasite harvest, 96 well plates containing confluent fibroblasts were set up with 1.33× the desired final drug concentrations in 150 μl of volume. This ensured that the addition of 50 μl of parasites would result in the desired 1× drug concentration of both pyrimethamine and DAHP. Wells designated as containing less than the maximum dosage of either drug (10 μg/ml pyrimethamine and 2.5 mM DAHP) received the equivalent volume of vehicle (ethanol for pyrimethamine, PBS for DAHP). Parasites were then force-lysed, filtered, and resuspended to a concentration of 5e5 parasites/ml. 50 μl of parasites were then added to each well, resulting in an infection of 2.5e4 parasites per well. Plates were then spun at 290 g for 5 min. Plates were then incubated for 3 days to allow for parasite plaquing. Plates were then washed with PBS followed by staining with crystal violet as conducted for plaque assays. Well staining was then quantified by plate reader at the 570 nm wavelength. Percent lysis metrics for each drug condition were normalized to wells containing parasites with no drugs (minimum staining) and wells containing no parasites (maximum staining). Drug curves were then generated and IC₅₀ values were calculated from biological triplicate experiments in GraphPad Prism 9.

### Plasmodium Cell Culture

The NF54 P. falciparum line was cultured and synchronized as described previously.^{28,29} Cultures were kept at 37° C. in RPMI-1640 medium supplemented with 25 mM HEPES, 100 μM hypoxanthine, 0.5% Albumax II, 24 mM sodium bicarbonate, 25 μg/mL gentamicin (Life Technologies, Carlsbad, CA 11021-045) and gassed with 5% CO₂, 1% O₂, and 94% N₂ mixture.

Dose-Response Assay in Plasmodium falciparum

A SYBR Green I-based assay was used to measure in vitro P. falciparum drug susceptibility.^{30,31} Ring-stage NF54 parasites were seeded at 1% hematocrit and 1% starting parasitemia in 384-well black clear-bottom plates containing test compounds (stocks of 10 mM pyrimethamine, 0.1 mM atovaquone, and 10 mM DHA in DMSO) plated in triplicate in 12-point serial dilutions for 72 h. The parasites were plated in complete RPMI 1640 media supplemented with 25 mM HEPES, 0.21% sodium bicarbonate, 50 mg/l hypoxanthine, and 0.5% Albumax II (Invitrogen) with or without 1 mM DAHP. Lysis buffer (0.16% w/v saponin, 1.6% Triton X-100, 5 mM EDTA, and 20 mM Tris-HCl, pH 7.4) with a 1:1000 dilution of SYBR Green I fluorescent dye (Invitrogen) was added for at least 12 h, and fluorescence readings were taken (excitation at 494 nm, emission at 530 nm) using a SpectraMax M5 (Molecular Devices, Sunnyvale, CA) plate reader. IC₅₀ values were calculated using a nonlinear regression curve fit in Graphpad Prism 9 in biological triplicate.

### Proliferation of Mouse Embryonic Fibroblasts in DAHP Treatment

Twenty-thousand immortalized C57BL/6J mouse embryonic fibroblasts were plated in a well of a 6-well plate and treated with 5 mM DAHP or PBS vehicle. The cells were allowed to grow for 48 hours, then trypsinized and counted. Population doublings were calculated as follows:

$$cell multiple = \frac{cell count at 48 hr}{input cell count}$$

$$population doubling = \frac{ln ( cell multiple )}{ln ( 2 )}$$

Generation of DD-BFD2, CST10-fLuc, pSAG1-nLuc Reporter Strain for Drug Screening

A pTub-DD-BFD2-HA_pSAG1-mNeonGreen construct was transfected into ME49ΔKu80ΔHX parasites with homology arms to an intergenic region of the parasite genome. This construct was co-transfected with a Cas9 plasmid containing a gRNA that targets this intergenic region, resulting in integration of the construct at the intended locus. The pSAG1-mNeonGreen was used to sort for successful integrants as mNeonGreen positive cells. A clonally-derived population was generated from this bulk transfection. A HiT vector targeting CST10 carrying a p2a-firefly luciferase reporter was transfected into this strain following previously described methods.³² This construct contained a chloramphenicol resistance cassette, allowing for selection of integrants by chloramphenicol selection. This bulk-selected strain was then transfected with a pSAG1-nanoLuciferase_pTUB-TdTomato construct targeted to another intergenic region. Sorting for TdTomato expression enriched for successful integrants. A clonally-derived population was then expanded from this bulk transfection and verified by sanger sequencing at the targeted loci.

### Firefly and Nano-Luciferase Assays

The Progema Nano-Glo Dual-Luciferase reporter kit was used to measure luciferase expression following the manufacturer's protocol. In these assays, five thousand reporter parasites were seeded into each well of a 96-well plate containing confluent human foreskin fibroblasts. Four hours after infection, the media was changed to include 3 μM shield-1 or a vehicle control to induce differentiation. At the indicated time points, shield-1 was removed from some wells, washed 3× with PBS, and replaced with media lacking shield-1 to yield bradyzoites in the absence of any exogenous active induction. Shield-1 is a compound having the following chemical structure:

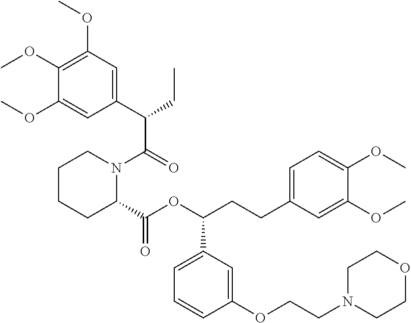 [SMILES: CC[C@H](C(=O)N1CCCC[C@H]1C(=O)O[C@H](CCc1ccc(OC)c(OC)c1)c1cccc(OCCN2CCOCC2)c1)c1cc(OC)c(OC)c(OC)c1]

Primer table

| Primer | | SEQ ID | | Usage |
|---|---|---|---|---|
| code | Primer name | NO: | Primer sequence | |
| CG545 | 5′UTRgRNA_TG | SEQ ID | tggggatgtcaagttGTGTTTTC | gRNA cloning |
| | GT1_253780 | NO: 1 | CGTGATCTTAACAgttttag | |
| | | | agctagaa | |
| CG546 | 3′UTRgRNA_TG | SEQ ID | tggggatgtcaagttGTTACCTC | gRNA cloning |
| | GT1_253780 | NO: 2 | TTCTTCTGCACGGgttttag | |
| | | | agctagaa | |
| CG561 | mNG_gch_ | SEQ ID | ACTTCTGGGTGAACCA | Amplify |
| | overhangs_ | NO: 3 | GCGGGAGGCGGGCGTG | mNeonGreen for |
| | 5UTR | | CAAAAGCAccatgcatgtccc | transfection |
| | | | gcgttcg | |
| CG562 | mNG_gch_ | SEQ ID | GGGAATGCTGTGAAGT | Amplify |
| | overhangs_ | NO: 4 | TCAGTTTGACATTCGGC | mNeonGreen for |
| | 3UTR | | GCCACAGgtgtcactgtagcctg | transfection |
| | | | ccag | |
| CG581 | TGGT1_253780_F | SEQ ID | CCCCACATAAAAGTCT | Verify gch |
| | | NO: 5 | GTTGGGCT | knockout |
| CG582 | TGGT1_253780_R | SEQ ID | GCATTGTGGGCTGTGA | Verify gch |
| | | NO: 6 | CCC | knockout |

### REFERENCES

1. Krishnan, A. et al. Functional and Computational Genomics Reveal Unprecedented Flexibility in Stage-Specific Toxoplasma Metabolism. Cell Host Microbe 27, 290-306.e11 (2020).
2. Tymoshenko, S. et al. Metabolic Needs and Capabilities of Toxoplasma gondii through Combined Computational and Experimental Analysis. PLOS Comput. Biol. 11, e1004261 (2015).
3. Blume, M. & Seeber, F. Metabolic interactions between Toxoplasma gondii and its host. F1000Res. 7, (2018).
4. Cantor, J. R. et al. Physiologic Medium Rewires Cellular Metabolism and Reveals Uric Acid as an Endogenous Inhibitor of UMP Synthase. Cell vol. 169 258-272.e17 (2017).
5. Folate synthesis in plants: The first step of the pterin branch is mediated by a unique bimodular GTP cyclohydrolase I. PNAS 99(19) 12489-12494 (2002).
6. Kümpornsin, K. et al. Biochemical and functional characterization of Plasmodium falciparum GTP cyclohydrolase I. Malar. J. 13, 150 (2014).
7. Thöny, B., Leimbacher, W., Blau, N., Harvie, A. & Heizmann, C. W. Hyperphenylalaninemia due to defects in tetrahydrobiopterin metabolism: molecular characterization of mutations in 6-pyruvoyl-tetrahydropterin synthase. Am. J. Hum. Genet. 54, 782-792 (1994).
8. Nzila, A., Ward, S. A., Marsh, K., Sims, P. F. G. & Hyde, J. E. Comparative folate metabolism in humans and malaria parasites (part I): pointers for malaria treatment from cancer chemotherapy. Trends Parasitol. 21, 292-298 (2005).
9. Nzila, A., Ward, S. A., Marsh, K., Sims, P. F. G. & Hyde, J. E. Comparative folate metabolism in humans and malaria parasites (part II): activities as yet untargeted or specific to Plasmodium. Trends Parasitol. 21, 334-339 (2005).
10. Krishnan, A., Kloehn, J., Lunghi, M. & Soldati-Favre, D. Vitamin and cofactor acquisition in apicomplexans: Synthesis versus salvage. J. Biol. Chem. 295, 701-714 (2020).
11. 11965-DMEM, high glucose-US.
12. Human Plasma-Like Medium (HPLM)-US.
13. Haldar, K., Bhattacharjee, S. & Safeukui, I. Drug resistance in Plasmodium. Nat. Rev. Microbiol. 16, 156-170 (2018).
14. Alday, P. H. & Doggett, J. S. Drugs in development for toxoplasmosis: advances, challenges, and current status. Drug Des. Devel. Ther. 11, 273-293 (2017).
15. Stepwise acquisition of pyrimethamine resistance in the malaria parasite. PNAS 106 (29) 12025-12030 (2009).
16. van Lenthe, M. et al. Markers of sulfadoxine-pyrimethamine resistance in Eastern Democratic Republic of Congo; implications for malaria chemoprevention. Malar. J. 18, 430 (2019).
17. Hyde, J. E. Exploring the folate pathway in Plasmodium falciparum. Acta Trop. 94, 191-206 (2005).
18. Cosentino, F. & Katusić, Z. S. Tetrahydrobiopterin and dysfunction of endothelial nitric oxide synthase in coronary arteries. Circulation 91, 139-144 (1995).
19. Mitchell, B. M., Dorrance, A. M. & Webb, R. C. GTP cyclohydrolase 1 inhibition attenuates vasodilation and increases blood pressure in rats. Am. J. Physiol. Heart Circ. Physiol. 285, H2165-70 (2003).
20. Pickert, G. et al. Inhibition of GTP cyclohydrolase reduces cancer pain in mice and enhances analgesic effects of morphine. J. Mol. Med. 90, 1473-1486 (2012).
21. Tegeder, I. et al. GTP cyclohydrolase and tetrahydrobiopterin regulate pain sensitivity and persistence. Nat. Med. 12, 1269-1277 (2006).
22. Dai, Y., Cui, J., Gan, P. & Li, W. Downregulation of tetrahydrobiopterin inhibits tumor angiogenesis in BALB/c-nu mice with hepatocellular carcinoma. Oncol. Rep. 36, 669-675 (2016).
23. Chen, L. et al. Paracrine effect of GTP cyclohydrolase and angiopoietin-1 interaction in stromal fibroblasts on tumor Tie2 activation and breast cancer growth. Oncotarget 7, 9353-9367 (2016).
24. Rembold, H. Hemmung des Crithidia-Wachstums durch 4-Amino-pyrimidine. Biological Chemistry 339, 258-259 (1964).
25. Xie, L., Smith, J. A. & Gross, S. S. GTP cyclohydrolase I inhibition by the prototypic inhibitor 2, 4-diamino-6-hydroxypyrimidine. Mechanisms and unanticipated role of GTP cyclohydrolase I feedback regulatory protein. J. Biol. Chem. 273, 21091-21098 (1998).
26. Kolinsky, M. A. & Gross, S. S. The mechanism of potent GTP cyclohydrolase I inhibition by 2,4-diamino-6-hydroxypyrimidine: requirement of the GTP cyclohydrolase I feedback regulatory protein. J. Biol. Chem. 279, 40677-40682 (2004).
27. Osei, M. et al. Amplification of GTP-cyclohydrolase 1 gene in Plasmodium falciparum isolates with the quadruple mutant of dihydrofolate reductase and dihydropteroate synthase genes in Ghana. PLOS One 13, e0204871 (2018).
28. Trager, W. & Jensen, J. B. Human malaria parasites in continuous culture. Science 193, 673-675 (1976).
29. Lambros, C. & Vanderberg, J. P. Synchronization of Plasmodium falciparum erythrocytic stages in culture. J. Parasitol. 65, 418-420 (1979).
30. Johnson, J. D. et al. Assessment and continued validation of the malaria SYBR green I-based fluorescence assay for use in malaria drug screening. Antimicrob. Agents Chemother. 51, 1926-1933 (2007).
31. Smilkstein, M., Sriwilaijaroen, N., Kelly, J. X., Wilairat, P. & Riscoe, M. Simple and inexpensive fluorescence-based technique for high-throughput antimalarial drug screening. Antimicrob. Agents Chemother. 48, 1803-1806 (2004).
32. Smith, T. A., Lopez-Perez, G. S., Herneisen, A. L., Shortt, E. & Lourido, S. Screening the Toxoplasma kinome with high-throughput tagging identifies a regulator of invasion and egress. Nat Microbiol 7, 868-881 (2022).
33. Fry, M. & Pudney, M. Site of action of the antimalarial hydroxynaphthoquinone, 2-[trans-4-(4′-chlorophenyl) cyclohexyl]-3-hydroxy-1,4-naphthoquinone (566C80). Biochem. Pharmacol. 43, 1545-1553 (1992).
34. Licon, M. H. et al. A positive feedback loop controls Toxoplasma chronic differentiation. Nat Microbiol (2023) doi: 10.1038/s41564-023-01358-2.

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of reducing viability of an apicomplexan parasite in a subject in need thereof, the method comprising administering an effective amount of an inhibitor of guanosine-5′-triphosphate cyclohydrolase I (GCH) to the subject.

2. The method of claim 1, wherein the inhibitor of GCH is 2,4-diamino-6-hydroxypyrimidine (DAHP).

3. The method of claim 1, wherein the inhibitor of GCH is 4-chloro-2,6-diaminopyrimidine.

4. The method of claim 1, wherein the inhibitor of GCH is 4-bromo-2,6-diaminopyrimidine.

5. The method of claim 1, wherein the inhibitor of GCH is 4-iodo-2,6-diaminopyrimidine.

6. The method of claim 1, wherein the inhibitor of GCH is 5-chloro-2,4-diamino-6-hydroxypyrimidine.

7. The method of claim 1, wherein the inhibitor of GCH is 5-bromo-2,4-diamino-6-hydroxypyrimidine.

8. The method of claim 1, wherein the inhibitor of GCH is 5-iodo-2,4-diamino-6-hydroxypyrimidine.

9. The method of claim 1, wherein the inhibitor of GCH is 2,4,5-triamino-6-hydroxypyrimidine.

10. The method of claim 1, wherein the inhibitor of GCH is guanine.

11. The method of claim 1, wherein the inhibitor of GCH is 8-bromoguanine.

12. The method of claim 1, wherein the inhibitor of GCH is 8-hydroxyguanine.

13. The method of claim 1, wherein the inhibitor of GCH is 8-methylguanine.

14. The method of claim 1, wherein the inhibitor of GCH is 8-mercaptoguanine.

15. The method of claim 1, wherein the inhibitor of GCH is 8-azaguanine.

16. The method of any one of claims 1-15, wherein the apicomplexan parasite is a Toxoplasma genus apicomplexan parasite.

17. The method of claim 16, wherein the apicomplexan parasite is Toxoplasma gondii.

18. The method of any one of claims 1-15, wherein the apicomplexan parasite is a Plasmodium genus apicomplexan parasite.

19. The method of claim 18, wherein the apicomplexan parasite is Plasmodium vivax, Plasmodium falciparum, Plasmodium malariae, Plasmodium ovale, or Plasmodium knowlesi.

20. The method of claim 18, wherein the apicomplexan parasite is Plasmodium falciparum.

21. The method of any one of claims 1-15, wherein the apicomplexan parasite is a Hammondia genus apicomplexan parasite.

22. The method of any one of claims 1-15, wherein the apicomplexan parasite is a Neospora genus apicomplexan parasite.

23. The method of claim 22, wherein the apicomplexan parasite is Neospora caninum.

24. The method of any one of claims 1-15, wherein the apicomplexan parasite is a Sarcocystis genus apicomplexan parasite.

25. The method of claim 24, wherein the apicomplexan parasite is Sarcocystis neurona.

26. The method of any one of claims 1-15, wherein the apicomplexan parasite is a Theileria genus apicomplexan parasite.

27. The method of any one of claims 1-15, wherein the apicomplexan parasite is an Eimeria genus apicomplexan parasite.

28. The method of any one of claims 1-15, wherein the apicomplexan parasite is a Babesia genus apicomplexan parasite.

29. The method of any one of claims 1-15, wherein the apicomplexan parasite is a Cryptosporidium genus apicomplexan parasite.

30. The method of any one of claims 1-15, further comprising administering an inhibitor of dihydrofolate reductase (DHFR) to the subject.

31. The method of claim 30, wherein the inhibitor of dihydrofolate reductase is pyrimethamine.

32. The method of claim 31, wherein the method reduces the ICso of pyrimethamine by at least 10%.

33. The method of claim 31, wherein the method reduces the IC₅₀ of pyrimethamine by at least 70%.

34. The method of any one of claims 1-15, wherein the apicomplexan parasite is resistant to pyrimethamine.

35. The method of any one of claims 1-15, further comprising administering an inhibitor of dihydropteroate synthase (DHPS) to the subject.

36. The method of claim 35, wherein the inhibitor of dihydropteroate synthase is sulfadiazine.

37. The method of claim 36, wherein the method reduces the IC₅₀ of sulfadiazine by at least 10%.

38. The method of claim 36, wherein the method reduces the IC₅₀ of sulfadiazine by at least 70%

39. The method of any one of claims 1-15, wherein the apicomplexan parasite is resistant to sulfadiazine.

40. The method of any one of claims 1-15, wherein the subject is a human.

41. The method of any one of claims 1-15, wherein the subject is an animal.

42. The method of claim 41, wherein the animal is a cat.

43. The method of claim 41, wherein the animal is a cattle.

44. The method of claim 41, wherein the animal is a pig.

45. The method of claim 41, wherein the animal is a chicken.

46. The method of claim 41, wherein the animal is a sheep.

47. The method of claim 41, wherein the animal is a goat.

48. The method of any one of claims 1-15, wherein the subject has toxoplasmosis.

49. The method of any one of claims 1-15, wherein the subject is at risk for toxoplasmosis.

50. The method of claim 49, wherein the subject is immunocompromised.

51. The method of any one of claims 1-15, wherein the subject has malaria.

52. The method of any one of claims 1-15, wherein the subject is at risk for malaria.

53. The method of claim 52, wherein the subject is immunocompromised.

54. The method of any one of claims 1-15, wherein the subject has babesiosis.

55. The method of any one of claims 1-15, wherein the subject is at risk for babesiosis.

56. The method of any one of claims 1-15, wherein the subject has cryptosporidiosis.

57. The method of any one of claims 1-15, wherein the subject is at risk for cryptosporidiosis.

58. The method of any one of claims 1-15, wherein the inhibitor of GCH is administered at a rate from 50 mg/kg/day to 300 mg/kg/day.

59. A method of identifying an agent that reduces viability of an apicomplexan parasite, the method comprising:
a) culturing an apicomplexan parasite in a cell culture;
b) adding an agent to the cell culture; and
c) detecting a concentration of one or more of 7,8-dihydroneopterin triphosphate, 6-pyruvoyl-tetrahydropterin, tetrahydrobiopterin, tetrahydrofolate, folate, dihydrofolate, or dihydrobiopterin in the cell culture after a period of time,
wherein a decrease in concentration of one or more of 7,8-dihydroneopterin triphosphate, 6-pyruvoyl-tetrahydropterin, tetrahydrobiopterin, tetrahydrofolate, folate, dihydrofolate, or dihydrobiopterin indicates that the agent reduces viability of the apicomplexan parasite.

60. The method of claim 59, wherein the cell culture is folate-poor.

61. The method of claim 59, wherein the cell culture is folate-rich.

62. The method of claim 59, wherein the cell culture is BH4-poor

63. The method of claim 59, wherein the cell culture is BH4-rich.

64. The method of claim 59, wherein the apicomplexan parasite does not express GCH.

65. The method of any one of claims 59-64, further comprising adding an antiparasitic compound to the cell culture, wherein reduction of the IC₅₀ of the antiparasitic agent by at least 10% indicates synergism of the agent and the antiparasitic agent.

66. The method of any one of claims 59-64, wherein the apicomplexan parasite is Toxoplasma gondii.

67. The method of claim 66, wherein the Toxoplasma gondii expresses Bradyzoite Formation Deficient 2 (BFD2) tagged with a degradation domain.

68. The method of claim 66, wherein the Toxoplasma gondii expresses firefly luciferase driven by fusion via a T2A peptide to CST10.

69. The method of claim 66, wherein the Toxoplasma gondii expresses nano-luciferase driven by SAG1.

70. The method of any one of claims 59-64, wherein the apicomplexan parasite is a Plasmodium genus apicomplexan parasite.

71. The method of claim 70, wherein the apicomplexan parasite is Plasmodium vivax, Plasmodium falciparum, Plasmodium malariae, Plasmodium ovale, or Plasmodium knowlesi.

72. The method of claim 70, wherein the apicomplexan parasite is Plasmodium falciparum.

73. The method of any one of claims 59-64, wherein the apicomplexan parasite is a Neospora genus apicomplexan parasite.

74. The method of claim 73, wherein the apicomplexan parasite is Neospora caninum.

75. The method of any one of claims 59-64, wherein the apicomplexan parasite is a Sarcocystis genus apicomplexan parasite.

76. The method of claim 75, wherein the apicomplexan parasite is Sarcocystis neurona.

77. The method of any one of claims 59-64, wherein the apicomplexan parasite is a Babesia genus apicomplexan parasite.

78. The method of any one of claims 59-64, further comprising administering an effective amount of the agent to a subject in need thereof.
